(19) United States
(12) Patent Application Publication
(10) Pub. No.: US 2021/0128051 A1
(43) Pub. Date: May 6, 2021

(54) MONITORING AUTONOMIC NERVOUS SYSTEM (ANS) ACTIVITIES OF SUBJECT FOR PREVENTIVE MEDICINE

(71) Applicants: Xiaoping LI, Fukuoka-shi (JP); Qian XIA, Fukuoka-shi (JP); Jessica LI, Fukuoka-shi (JP); Joanna Le Xi LI, Fukuoka-shi (JP)

(72) Inventors: Xiaoping LI, Fukuoka-shi (JP); Qian XIA, Fukuoka-shi (JP); Jessica LI, Fukuoka-shi (JP); Joanna Le Xi LI, Fukuoka-shi (JP)

(21) Appl. No.: 17/091,380

(22) Filed: Nov. 6, 2020

Related U.S. Application Data
(60) Provisional application No. 62/931,447, filed on Nov. 6, 2019.

(51) Int. Cl. A61B 5/00; A61B 5/0537

(52) U.S. Cl. CPC A61B 5/4035; A61B 5/4266; A61B 5/4809; A61B 5/7264; A61B 5/6823; A61B 5/407; A61B 5/6824; A61B 5/0537

(57) ABSTRACT

Disclosed is a system for monitoring activities of Autonomic Nervous System (ANS) for preventive medicine comprising: a sensing device configured to sense an involuntary control signal from the ANS of a subject in sleep; and an analyzing device configured to analyze the sensed involuntary control signal received from the sensing device to evaluate a dynamic health condition of the subject, and output the evaluated dynamic health condition.

### CROSS-REFERENCE TO RELATED PATENT APPLICATION

This application claims the benefit of priority under 35 USC § 119(e) of U.S. provisional patent application No. 62/931,447 filed 6 Nov. 2019, the contents of which is incorporated herein by reference in their entirety.

### TECHNICAL FIELD

The present disclosure generally relates to monitoring Autonomic Nervous System (ANS) activities of a subject for preventive medicine.

### BACKGROUND

There is a need for preventive medicine, in which people know their body dynamic healthy conditions and receive instructions as well as needed cares to prevent illness from happening to them. This requires a system that detects a person's body dynamic health conditions daily, analyzing the conditions, and provide the person with the results, healthcare instructions and necessary cares.

The human body is formed by 12 systems: the cardiovascular system, the digestive system, the endocrine system, the excretory system, the immune system, the hematopoietic system, the lymphatic system, the muscular & skeletal system, the central nervous system, the reproductive system, respiratory system, and the sensory system. Of the 12 systems the central nervous system controls the other systems, with its two kinds of control signal: voluntary control signals from the motor nervous system or cerebellum, and involuntary control signals from the autonomic nervous system. The voluntary control signals control the muscular & skeletal system as well as the sensory system for the body actions to external. The involuntary control signals control all the systems for the internal activities, including all the health conditions, of the body.

By monitoring the involuntary control signals, the internal activities and the dynamic health conditions of all the body systems may be monitored. However, both the voluntary control signals and involuntary control signals are electric impulses, with the voluntary control signals being normally many times larger in terms of the electric signal intensity, and normally both of the voluntary control signals and involuntary control signals are descended from the brain to the body, thus the involuntary control signals measured in a mixture with the voluntary control signals are hardly detected.

### SUMMARY

The present disclosure is generally to provide at least a solution for monitoring a person's body dynamic conditions, analyzing the dynamic conditions, and/or providing the person with the results, instructions and/or healthcare if necessary, preventing illnesses from happening.

The present disclosure provides a system having brain modulator modulating a person's brain to block the descending of the voluntary control signal while the measurement of the involuntary control signals are been carried, and further, having the measured the involuntary control signals analyzed for the dynamic health conditions of the body systems and feedback to the person with the results and healthcare instructions when necessary.

As an aspect of the present disclosure, provided is a system of sensing the involuntary control signals of the autonomic nervous system for preventive medicine, to sense the involuntary control signals of the autonomic nervous system for sensing the dynamic conditions of each of the body systems.

In certain embodiments, the involuntary control signals of the autonomic nervous system for sensing the dynamic conditions of each of the body systems is sensed by: 1. putting the brain of a person into a deep sleep mode such that the voluntary control signals of the somatic nervous system is blocked from coming out the brain to other body systems such that the involuntary control signals of the autonomic nervous system in controlling each of the body systems can be sensed with the interference of the voluntary control signals, 2. the involuntary control signals of the autonomic nervous system of the person are sensed by settings of signal acquisition distributed on the body, 3. the involuntary control signals of the autonomic nervous system sensed by the settings of signal acquisition distributed on the body are transmitted to a signal processing system containing a database for storing all incoming signal data, a signal analyzer for analyzing the dynamic conditions of each of the body systems from received involuntary control signals of the autonomic nervous system, and a communicator for providing the sensed results of dynamic conditions of each of the body systems to the person as well as any necessary cares to the person.

In certain embodiments, the brain of a person is put into a deep sleep mode such that the voluntary control signals of the somatic nervous system is blocked from descending from the brain to other body systems, by a physical device that modulates the brain into a deep sleep mode.

In certain embodiments, electric potential apparatus is set on one or more parts of the body to sense the involuntary control signals of the autonomic nervous system for a body system.

In certain embodiments, artificial intelligence is fully equipped in the signal processing system, including the database, signal analyzer, and communicator.

It should be noted that, the above illustration only shows a summary of the technical solutions of the invention for more clearly understanding the technical means of the invention and hence implementing the invention in accordance with the contents of the disclosure. In order to make the above and other objects, features and advantages more apparent, specific implementation of the invention will be illustrated below by examples.

### BRIEF DESCRIPTION OF THE DRAWINGS

One of ordinary skills in the art may understand the advantages and benefits described herein and other advantages and benefits by reading the detailed description of the exemplary embodiments below. The drawings are merely provided for illustrating some exemplary embodiments, rather than being regarded as limiting the invention. Moreover, throughout the drawings, the same reference signs are used to represent the same elements. In the drawings:

FIG. 1 shows an experimental result of when the immune system fights against viral infection, the immune cells destroy cells infected with viruses causing higher body temperature condition; and during deep sleep of the brain, when the descending of the voluntary control signals are blocked as a result of sleep hormone fully released in the brain, such an involuntary control signals can be clearly measured by placing electrodes on any part of the body where sweat glands are in the skin.

FIG. 2 is a schematic drawing of the signal acquisition module of the system of sensing the involuntary control signals of the autonomic nervous system for preventive medicine in the present disclosure, having a deep sleep modulator acting on the brain and a setting of multiple electric sensors attached to the body parts.

FIG. 3 is a schematic drawing of the whole system of sensing the involuntary control signals of the autonomic nervous system for preventive medicine in the present disclosure, having a signal acquisition module and a signal processing and communication module.

### DETAILED DESCRIPTION

Exemplary embodiments of the present disclosure will be described in more detail below with reference to the drawings. Although exemplary embodiments of this disclosure are shown in the drawings, it should be understood that this disclosure may be implemented in various forms, rather than being limited to the embodiments illustrated herein. On the contrary, these embodiments are provided for more thoroughly understanding this disclosure and fully conveying the scope of this disclosure to one skilled in the art.

It should be understood that, in the present disclosure, terms such as “include” or “have”, etc., intend to indicate the existence of a feature, a digit, a step, a behavior, a component, a part disclosed in the present disclosure or a combination thereof, without excluding the possibility of the existence of one or more other features, digits, steps, behaviors, components, parts or combinations thereof.

The invention will be illustrated in detail below referring to the drawings and in conjunction with the embodiments.

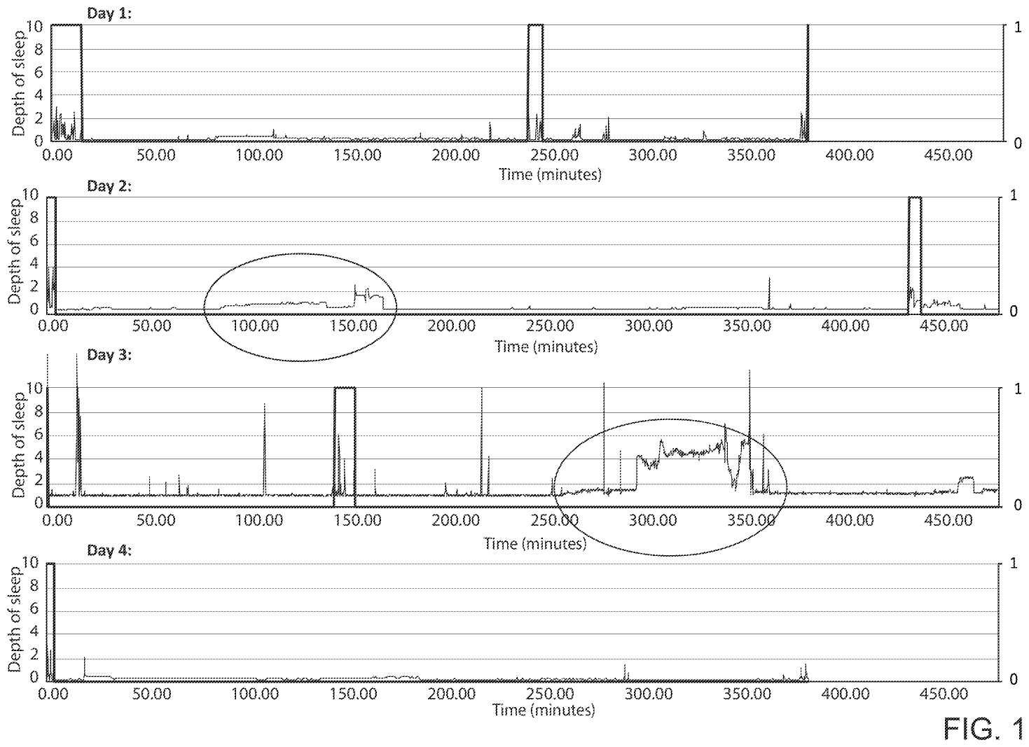

FIG. 1 shows an experimental result of when the immune system fights against viral infection, the immune cells destroy cells infected with viruses causing higher body temperature condition; and during deep sleep of the brain, when the descending of the voluntary control signals are blocked as a result of sleep hormone fully released in the brain, such an involuntary control signals can be clearly measured by placing electrodes on any part of the body where sweat glands are in the skin.

Particularly, when the immune system fights against viral infection, the immune cells, such as the CD8+ cytotoxic T-cells, is able to destroy cells infected with viruses and even tumor cells, meanwhile, a higher body temperature (like one achieved in a fever) raises, which means a condition of greater body response against infection. Accompanying with such a body condition is that the brain descends involuntary control signal controlling the sweat glands throughout the body to activate, preventing overheating of the body by releasing sweat out of the body. During deep sleep of the brain, when the descending of the voluntary control signals are blocked as a result of sleep hormone fully released in the brain, such an involuntary control signals can be clearly measured by placing an electric potential sensing apparatus on any part of the body where sweat glands are in the skin. FIG. 1 shows such an experimental result. In Day 1, the subject stayed in house during the day, minimizing the chances of viral infection. Then at night during a 6-hour deep sleep, no signals for activating sweat glands were detected. In Day 2, the subject went out and maximized the chances of getting viruses into his airways. Then at night during an 8-hour deep sleep, signals for activating sweat glands were detected, indicating a battle of immune cells fighting against viral infection occurred in the body. In Day 3, the subject stayed in house during the day, minimizing the chances of viral infection. Then still, at night during an 8-hour deep sleep, significant signals for activating sweat glands were detected, indicating a battle of immune cells fighting against viral infection continuing from D2 occurred in the body. In Day 4, the subject stayed in house during the day, minimizing the chances of viral infection. Then at night during with an 8-hour deep sleep, no signals for activating sweat glands were detected, indicating the battle of immune cells fighting against viral infection occurred in the body had already completed in Day 3.

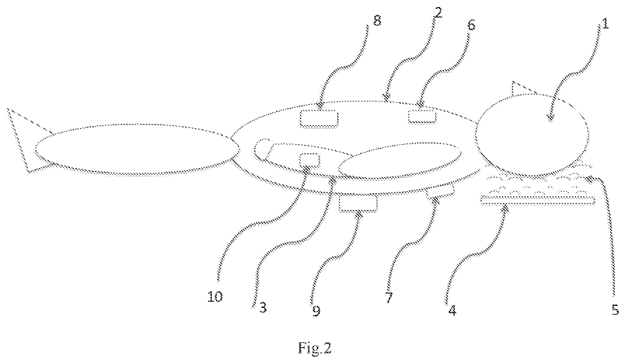

In an embodiment, as shown in FIG. 2, as an example, a signal acquisition module of the system of sensing the involuntary control signals of the autonomic nervous system with regard to the cardiovascular system, the digestive system, and the immune system are presented. The subject lies down having the head 1 placed above a sleep induction device 4, with the head locating in the inductive electric field 5 and receiving modulation for sleep hormone fully release in the brain such that the brain descending of voluntary control signals is blocked. The sensing of the involuntary control signals with regard to the cardiovascular system, the digestive system, and the immune system are through electric potential sensing apparatus 6 and 7, electric potential sensing apparatus 8 and 9, and electric potential sensing apparatus 10, respectively, where apparatus 6 is placed near the heart for sensing the involuntary control signals at the cardiovascular system, apparatus 7 is placed near the spinal nerve exit T1-T4 to sense the involuntary control signal towards the heart, apparatus 8 is placed at the stomach for sensing the involuntary control signals at the digestive system, apparatus 9 is placed near the spinal nerve exit T9-T12 to sense the involuntary control signals towards the digestive system, and apparatus 10 is placed on the arm 3 to sense the sweat gland activation in relation to the immune system.

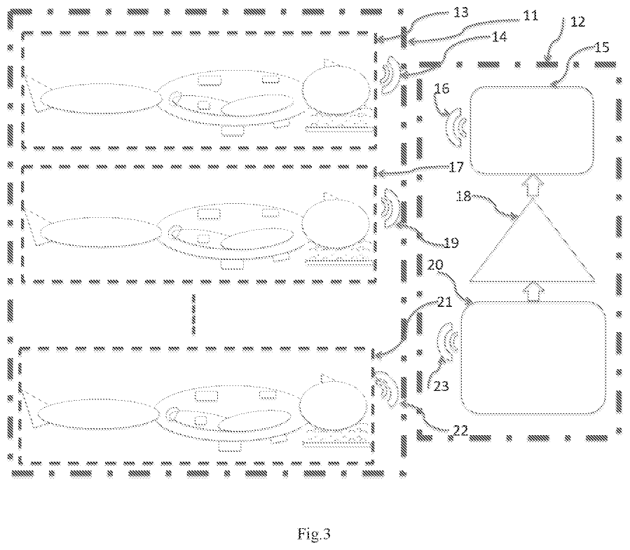

In an embodiment, as shown in FIG. 3, the whole system of sensing the involuntary control signals of the autonomic nervous system for preventive medicine is formed by the signal acquisition module 11 and the signal processing and communicator module 12 that is fully equipped with artificial intelligence. The signal acquisition module 11 consists of a number of users 13, 17, . . . , 21 of the system, each transmit its sensed signal data via the wireless transmitter 14, 19, . . . , 22, respectively, to the signal processing and communicator module 12, received via the wireless receiver 23, into the database of involuntary control signals from users No. 1 . . . No. n, 20, then been passed to the signal analyzer 18, with the results been passed to the Communicator 15, which transmits the analyzed results, instructions and cares to the users via wireless transmitter 16.

Finally, it should be noted that, the above embodiments are only provided for illustrating, rather than limiting, the technical solutions of the invention; although detailed illustration of the invention has being given referring to the above embodiments, it should be understood by one of ordinary skills in the art that modifications may be made on the technical solutions recorded in each of the above embodiment, or equivalent substitutions may be made on a part of the technical features thereof, without departing from the concept and scope of the technical solutions in each embodiment of the invention.

What is claimed is:

1. A system for monitoring activities of Autonomic Nervous System (ANS) for preventive medicine, comprising:
a sensing device configured to sense an involuntary control signal from the ANS of a subject in sleep; and
an analyzing device configured to analyze the sensed involuntary control signal received from the sensing device to evaluate a dynamic health condition of the subject, and output the evaluated dynamic health condition.

2. The system according to claim 1, wherein the system further comprises a sleep induction device configured to induce the subject to fall in sleep, and the sensing device is configured to sense the involuntary control signal from the ANS of the subject after the subject is induced to fall in sleep by the sleep induction device.

3. The system according to claim 1, wherein the sensing device comprises at least one measurement unit configured to measure at least one electric potential on skin near at least one body part of the subject.

4. The system according to claim 3, wherein the at least one body part comprises at least one of a heart, a stomach, a spinal nerve exit, and an arm.

5. The system according to claim 1, wherein the analyzing device comprises a database unit configured to store data at least necessary for evaluating the dynamic health condition, an analyzing unit configured to analyze the sensed involuntary control signal received from the sensing device to evaluate the dynamic health condition of the subject, and a communication unit configured to output the evaluated dynamic health condition.

6. The system according to claim 5, wherein the analyzing unit is equipped with artificial intelligence.
